(19) United States
(12) Patent Application Publication
(10) Pub. No.: US 2020/0345013 A1
(43) Pub. Date: Nov. 5, 2020

(54) ANTI-ADHERENT BOTANICAL COMPOSITIONS

(71) Applicant: Kimberly-Clark Worldwide, Inc., Neenah, WI (US)

(72) Inventors: David W. Koenig, Menasha, WI (US); Kathleen C. Engelbrecht, Kaukauna, WI (US); Amy L. Vanden Heuvel, Hortonville, WI (US); Vinod Chaudhary, Appleton, WI (US); Scott W. Wenzel, Neenah, WI (US); Divesh Bhatt, Marietta, GA (US); Yang Huang, Shanghai (CN); Aimin He, Alpharetta, GA (US)

(21) Appl. No.: 16/935,617

(22) Filed: Jul. 22, 2020

Related U.S. Application Data
(63) Continuation of application No. 15/521,468, filed on Apr. 24, 2017.

(51) Int. Cl. A01N 65/00; A01N 31/16; A01N 43/32; A01N 45/00; A01N 65/08; A01N 65/20; A01N 65/30; A01N 65/40; A61K 36/708; A61K 8/02; A61K 36/48; A61K 36/8962; A61K 36/16; A61K 8/49; A61K 8/34; A61K 36/481; A61K 36/77; A61Q 17/00; A61K 36/482; A61K 8/9771; A61K 8/9789; A61K 8/9794; A01N 25/08; A01N 65/24

(52) U.S. Cl. CPC A01N 65/00; A01N 65/24; A01N 43/32; A01N 45/00; A01N 65/08; A01N 65/20; A01N 65/30; A01N 65/40; A61K 36/708; A61K 8/0208; A61K 36/48; A61K 36/8962; A61K 36/16; A61K 8/498; A61K 8/347; A61K 36/481; A61K 36/77; A61Q 17/00; A61K 36/482; A61K 8/9771; A61K 8/9789; A61K 8/9794; A01N 25/08; A01N 31/16

(57) ABSTRACT

A composition for inhibiting the attachment of microbes to an animate or inanimate surface is disclosed. The composition includes a carrier and an effective amount of an anti-adherent agent. The anti-adherent agents include botanical agents such as Soybean extract, Horse chestnut extract, Gypenoside, Resveratrol, Astragalus extract, Rhubarb root extract, Allium cepa extract, Cassia seed extract, Ginkgo biloba extract, and Silymarin, and combinations thereof. The efficacy of the botanical agents is microbe and surface dependent. Various delivery vehicles, including a wipe, may be used to deliver the composition to the surface.

### TECHNICAL FIELD

Disclosed is a composition with anti-adherent properties. More specifically, disclosed is are compositions that include an anti-adherent agent that does not adhere to certain infectious agents, including but not limited to Gram-negative, Gram-positive bacteria, yeast and virus. The compositions may be applied to or incorporated into articles such as wipes, or into ointments, lotions, creams, salves, aerosols, gels, suspensions, sprays, foams, washes, or the like.

### BACKGROUND OF THE DISCLOSURE

Communicable human infections pass from person to person through various means such as food, surfaces and hands. For example, in the United States, foodborne pathogens alone cause an estimated 76 million cases of illness, 325,000 hospitalizations and 5,000 deaths per year. This results in the spending or loss of several billion dollars due to absenteeism, cost of medication, and hospitalization.

Foodborne pathogens are typically a result of poor cleaning of hands and surfaces on which food is prepared. In fact, the kitchen is one of the most contaminated sites in the home. High fecal and coliform concentrations can be found in sponges, dishcloths, and the kitchen sink. Of course, there are other pathogens lurking elsewhere in the home, at the office, and in public places such as public bathrooms, restaurants, malls, theaters, health-care facilities, etc. Such pathogens include bacteria, protein, active enzymes, viruses, and many other microbes that can lead to health problems such as bacterial infections.

There are products used today that are used to clean skin and hard surfaces, such as soaps, hand sanitizers, sprays and wipes. However, even the most diligent efforts to keep clean can be hindered by factors such as surface topography, the presence of hair, and the like. These factors can cause pathogens to better adhere to a surface. Other limiting factors include skin sensitivity due to the handling of cleaning products or the application thereof.

There remains a need for compositions that can be applied to surfaces or incorporated into articles, wherein the compositions prevent the adherence of pathogens. Desirably, the compositions are skin friendly, cost effective, and convenient to use.

### SUMMARY OF THE DISCLOSURE

In one aspect of the disclosure there is a composition for inhibiting the attachment of a microbe to a surface. The composition includes a carrier and an effective amount of botanical agents. The agents may be selected from Soybean extract, Horse chestnut extract, Gypenoside, Resveratrol, Astragalus extract, Rhubarb root extract, Allium cepa extract, Cassia seed extract, Ginkgo biloba extract, Silymarin, and combinations thereof.

In another aspect of the disclosure is a composition for inhibiting the attachment of a microbe to a surface, the composition including a hydrophilic carrier and an effective amount of a botanical agent selected from Soybean extract, Horse chestnut extract, Gypenoside, Resveratrol, Astragalus extract, Rhubarb root extract, Allium cepa extract, Cassia seed extract, Ginkgo biloba extract, and Silymarin, and combinations thereof. The botanical agent reduces the attachment of a microbe to a test surface by at least 0.5 Log of bacteria.

In yet another aspect of the disclosure is a wipe. The wipe includes a nonwoven substrate and an anti-adherent composition comprising 0.01% to 20% (by total weight of composition) of a botanical agent. The botanical agent is selected from Soybean extract, Horse chestnut extract, Gypenoside, Resveratrol, Astragalus extract, Rhubarb root extract, Allium cepa extract, Cassia seed extract, Ginkgo biloba extract, Silymarin, and combinations thereof. The composition further includes a hydrophilic carrier. The composition reduces the adherence of Staphyloccus aureus on a surface.

### DETAILED DESCRIPTION OF THE DISCLOSURE

The present disclosure is directed to an anti-adherent composition containing an anti-adherent agent and a carrier. The composition may be applied to a biotic or abiotic surface in the form of a liquid, gel, or foam; or incorporated into a wash. In addition, the composition may be applied to a biotic or abiotic surface with a vehicle such as a wipe.

The anti-adherent composition may be used on biotic surfaces such as skin or plants, or abiotic surfaces such as food prep surfaces; hospital and clinic surfaces; household surfaces; automotive, train, ship and aircraft surfaces; and the like, as long as the surface is compatible with the ingredients of the composition.

According to the High Throughput Anti-adherence Test Method, infra, the anti-adherent composition reduces adherence to Gram-negative and Gram-positive bacteria by at least 0.5 Log, or by at least 0.9 Log, or by at least by 1 Log.

Anti-adherent agents suitable for use in the present disclosure include botanical agents from Soybean extract, Horse chestnut extract, Gypenoside, Resveratrol, Astragalus extract, Rhubarb root extract, Allium cepa extract, Cassia seed extract, Ginkgo biloba extract and Silymarin (see Table 1 for the plant sources). As shown herein, the botanical agents have different degrees of anti-adherency with respect to Gram-negative bacteria, Gram-positive bacteria, yeast and a virus. These botanical agents perform adequately and vary in anti-adherency to microbes. As shown herein, selection of botanical extracts as anti-adherent agents is dependent on the microbes of interest and the end use of the anti-adherent composition.

TABLE 1
Anti-Adherent Agents

| Agent * | Plant Source |
|---|---|
| Soybean extract | Glycinemax |
| Horse chestnut extract | Aesculushippocastanum |
| Gypenoside | Gynostemmapentaphylla |
| Resveratrol | Polygonumcuspidatum |
| Astragalus extract | Astragalusmembranaceus |
| Rhubarb root extract | Rheum officinale |
| Allium cepa extract | Allium cepa |
| Cassia seed extract | Semen Cassiaeobtusifoliae |
| Ginkgo biloba extract | Ginkgo biloba |
| Silymarin | Silybummarianum |

* Manufacturer: Acetar Bio-Tech Inc., Xi'An, China

The anti-adherent compositions of the present disclosure can be suitably made with an anti-adherent agent in an amount of from about 0.01% (by the total weight of the composition), to about 20% (by total weight of the composition), or from about 0.05% (by total weight of the composition) to about 15% (by total weight of the composition), or from about 0.1% (by total weight of the composition) to about 10% (by total weight of the composition).

### Carriers

The anti-adherent compositions of the present disclosure may be formulated with one or more conventional and compatible carrier materials. The anti-adherent composition may take a variety of forms including, without limitation, aqueous solutions, gels, balms, lotions, suspensions, creams, milks, salves, ointments, sprays, emulsions, oils, resins, foams, solid sticks, aerosols, and the like. Carrier materials suitable for use in the instant disclosure include those well-known for use in the cosmetic and medical arts as a basis for ointments, lotions, creams, salves, aerosols, gels, suspensions, sprays, foams, washes, and the like, and may be used in their established levels.

Non-limiting examples of suitable carrier materials include water, emollients, humectants, polyols, surfactants, esters, silicones, clays, and other pharmaceutically acceptable carrier materials.

In one embodiment, the anti-adherent compositions can optionally include one or more emollients, which typically acts to soften, soothe, and otherwise lubricate and/or moisturize the skin. Suitable emollients that can be incorporated into the compositions include oils such as alkyl dimethicones, alkyl methicones, alkyldimethiconecopolyols, phenyl silicones, alkyl trimethylsilanes, dimethicone, dimethicone crosspolymers, cyclomethicone, lanolin and its derivatives, fatty esters, glycerol esters and derivatives, propylene glycol esters and derivatives, alkoxylated carboxylic acids, alkoxylated alcohols, fatty alcohols, and combinations thereof. These ingredients may be encapsulated or the like to prevent them from negatively affecting the anti-adherent agent. For instance, adding oil to a cylcodextrin prior to adding it to the anti-adherent composition.

The anti-adherent compositions may include one or more emollient in an amount of from about 0.01% (by total weight of the composition) to about 20% (by total weight of the composition), or from about 0.05% (by total weight of the composition) to about 10% (by total weight of the composition), or from about 0.10% (by total weight of the composition) to about 5% (by total weight of the composition).

In another embodiment the anti-adherent compositions may include one or more esters. The esters maybe selected from cetylpalmitate, stearyl palmitate, cetyl stearate, isopropyl laurate, isopropyl myristate, isopropyl palmitate, and combinations thereof. The fatty alcohols include octyldodecanol, lauryl, myristyl, cetyl, stearyl, behenyl alcohol, and combinations thereof. Ethers such as eucalyptol, ceterarylglucoside, dimethyl isosorbic polyglyceryl-3 cetyl ether, polyglyceryl-3 decyltetradecanol, propylene glycol myristyl ether, and combinations thereof can also suitably be used as emollients. Other suitable ester compounds for use in the anti-adherent compositions or the present disclosure are listed in the International Cosmetic Ingredient Dictionary and Handbook, 11th Edition, CTFA, (January, 2006) ISBN-10: 1882621360, ISBN-13: 978-1882621361, and in the 2007 Cosmetic Bench Reference, Allured Pub. Corporation (Jul. 15, 2007) ISBN 10.1932633278, ISBN-13: 978-1932633276, both of which are incorporated by reference herein to the extent they are consistent herewith.

Humectants that are suitable as carriers in the anti-adherent compositions of the present disclosure include, for example, glycerin, glycerin derivatives, hyaluronic acid, hyaluronic acid derivatives, betaine, betaine derivatives amino acids, amino acid derivatives, glycosaminoglycans, glycols, polyols, sugars, sugar alcohols, hydrogenated starch hydrolysates, hydroxy acids, hydroxy acid derivatives, salts of PCA and the like, and combinations thereof. Specific examples of suitable humectants include honey, sorbitol, hyaluronic acid, sodium hyaluronate, betaine, lactic acid, citric acid, sodium citrate, glycolic acid, sodium glycolate, sodium lactate, urea, propylene glycol, butylene glycol, pentylene glycol, ethoxydiglycol, methyl gluceth-10, methyl gluceth-20, polyethylene glycols (as listed in the International Cosmetic Ingredient Dictionary and Handbook such as PEG-2 through PEG 10), propanediol, xylitol, maltitol, or combinations thereof. Humectants are beneficial in that they prevent or reduce the chance that the anti-adherent film, formed after the anti-adherent agent is applied to a surface, will crack.

The anti-adherent compositions of the disclosure may include one or more humectants in an amount of about 0.01% (by total weight of the composition) to about 20% (by total weight of the composition), or about 0.05% (by total weight of the composition) to about 10% by total weight of the composition), or about 0.1% (by total weight of the composition) to about 5.0% (by total weight of the composition).

The anti-adherent compositions may include water. For instance, where the anti-adherent composition is a wetting composition, such as described below for use with a wet wipe, the composition will typically comprise water. The anti-adherent compositions can suitably comprise water in an amount of from about 0.01% (by total weight of the composition) to about 99.98% (by total weight of the composition), or from about 0.05% (by total weight of the composition) to about 95% (by total weight of the composition), or from about 0.10% (by total weight of the composition) to about 90% (by total weight of the composition).

In an embodiment where the anti-adherent composition serves as a wash (e.g. shampoo; surface cleanser; or hand, face, or body wash), the anti-adherent composition will include one or more surfactants. These may be selected from anionic, cationic, zwitterionic, nonionic and amphoteric surfactants. Amounts may range from 0.1 to 30%, or from 1 to 20%, or from 3 to 15% by total weight of the total composition.

Suitable anionic surfactants include, but are not limited to, C₈ to C₂₂ alkane sulfates, ether sulfates and sulfonates. Among the suitable sulfonates are primary C₈ to C₂₂ alkane sulfonate, primary C₈ to C₂₂ alkane disulfonate, C₈ to C₂₂ alkene sulfonate, C₈ to C₂₂hydroxyalkanesulfonate or alkyl glyceryl ether sulfonate. Specific examples of anionic surfactants include ammonium lauryl sulfate, ammonium laureth sulfate, triethylamine lauryl sulfate, triethylaminelaureth sulfate, triethanolamine lauryl sulfate, triethanolaminelaureth sulfate, monoethanolamine lauryl sulfate, monoethanolaminelaureth sulfate, diethanolamine lauryl sulfate, diethanolaminelaureth sulfate, lauricmonoglyceride sodium sulfate, sodium lauryl sulfate, sodium laureth sulfate, potassium laureth sulfate, sodium lauryl sarcosinate, sodium lauroylsarcosinate, potassium lauryl sulfate, sodium trideceth sulfate, sodium methyl lauroyltaurate, sodium lauroylisethionate, sodium laurethsulfosuccinate, sodium lauroylsulfosuccinate, sodium tridecyl benzene sulfonate, sodium dodecyl benzene sulfonate, sodium lauryl amphoacetate and mixtures thereof. Other anionic surfactants include the C₈ to C₂₂ acyl glycinate salts. Suitable glycinate salts include sodium cocoylglycinate, potassium cocoylglycinate, sodium lauroylglycinate, potassium lauroylglycinate, sodium myristoylglycinate, potassium myristoylglycinate, sodium palmitoylglycinate, potassium palmitoylglycinate, sodium stearoylglycinate, potassium stearoylglycinate, ammonium cocoylglycinate and mixtures thereof. Cationic counterions to form the salt of the glycinate may be selected from sodium, potassium, ammonium, alkanolammonium and mixtures of these cations.

Suitable cationic surfactants include, but are not limited to alkyl dimethylamines, alkyl amidopropylamines, alkyl imidazoline derivatives, quaternised amine ethoxylates, and quaternary ammonium compounds.

Suitable zwitterionic surfactants include, for example, alkyl amine oxides, silicone amine oxides, and combinations thereof. Specific examples of suitable zwitterionic surfactants include, for example, 4-[N,N-di(2-hydroxyethyl)-N-octadecylammonio]-butane-1-carboxylate, S—[S-3-hydroxypropyl-S-hexadecylsulfonio]-3-hydroxypentane-1-sulfate, 3-[P,P-diethyl-P-3,6,9-trioxatetradexopcylphosphonio]-2-hydroxypropane-1-phosphate, 3-[N,N-dipropyl-N-3-dodecoxy-2-hydroxypropylammonio]-propane-1-phosphonate, 3-(N,N-dimethyl-N-hexadecylammonio)propane-1-sulfonate, 3-(N,N-dimethyl-N-hexadecylammonio)-2-hydroxypropane-1-sulfonate, 4-[N,N-di(2-hydroxyethyl)-N-(2-hydroxydodecyl)ammonio]-butane-1-carboxylate, 3-[S-ethyl-S-(3-dodecoxy-2-hydroxypropyl)sulfonio]-propane-1-phosphate, 3-[P,P-dimethyl-P-dodecylphosphonio]-propane-1-phosphonate, 5-[N,N-di(3-hydroxypropyl)-N-hexadecylammonio]-2-hydroxy-pentane-1-sulfate, and combinations thereof.

Suitable nonionic surfactants include, but are not limited to, alcohols, acids, amides or alkyl phenols reacted with alkylene oxides, especially ethylene oxide either alone or with propylene oxide. Specific nonionics are C₆ to C₂₂ alkyl phenols-ethylene oxide condensates, the condensation products of C₈ to C₁₃ aliphatic primary or secondary linear or branched alcohols with ethylene oxide, and products made by condensation of ethylene oxide with the reaction products of propylene oxide and ethylenediamine. Other nonionics include long chain tertiary amine oxides, long chain tertiary phosphine oxides and dialkylsulphoxides, alkyl polysaccharides, amine oxides, block copolymers, castor oil ethoxylates, ceto-oleyl alcohol ethoxylates, ceto-stearyl alcohol ethoxylates, decyl alcohol ethoxylates, dinonyl phenol ethoxylates, dodecyl phenol ethoxylates, end-capped ethoxylates, ether amine derivatives, ethoxylated alkanolamides, ethylene glycol esters, fatty acid alkanolamides, fatty alcohol alkoxylates, lauryl alcohol ethoxylates, mono-branched alcohol ethoxylates, natural alcohol ethoxylates, nonyl phenol ethoxylates, octyl phenol ethoxylates, oleyl amine ethoxylates, random copolymer alkoxylates, sorbitan ester ethoxylates, stearic acid ethoxylates, stearyl amine ethoxylates, synthetic alcohol ethoxylates, tall oil fatty acid ethoxylates, tallow amine ethoxylates and tridtridecanolethoxylates.

Suitable amphoteric surfactants include, but are not limited to, derivatives of aliphatic quaternary ammonium, phosphonium, and sulfonium compounds, in which the aliphatic radicals can be straight or branched chain, and wherein one of the aliphatic substituents contains from about 8 to about 18 carbon atoms and one substituent contains an anionic group, e.g., carboxy, sulfonate, sulfate, phosphate, or phosphonate. Illustrative amnphoterics are coco dimethyl carboxymethyl betaine, cocoamidopropyl betaine, cocobetaine, oleyl betaine, cetyl dimethyl carboxymethyl betaine, lauryl bis-(2-hydroxyethyl) carboxymethyl betaine, stearyl bis-(2-hydroxypropyl) carboxymethyl betaine, oleyl dimethyl gamma-carboxypropyl betaine, lauryl bis-(2-hydroxypropyl)alpha-carboxyethyl betaine, cocoamphoacetates, and mixtures thereof. The sulfobetaines may include stearyl dimethyl sulfopropyl betaine, lauryl dimethyl sulfoethyl betaine, lauryl bis-(2-hydroxyethyl) sulfopropyl betaine and mixtures thereof.

### Rheology Modifier

Optionally, one or more rheology modifiers, such as thickeners, may be added to the anti-adherent compositions. Suitable rheology modifiers are compatible with the anti-adherent agent. As used herein, “compatible” refers to a compound that, when mixed with the anti-adherent agent, does not adversely affect the anti-adherent properties of same.

A thickening system is used in the anti-adherent compositions to adjust the viscosity and stability of the compositions. Specifically, thickening systems prevent the composition from running off of the hands or body during dispensing and use of the composition. When the anti-adherent composition is used with a wipe product, a thicker formulation can be used to prevent the composition from migrating from the wipe substrate.

The thickening system should be compatible with the compounds used in the present disclosure; that is, the thickening system, when used in combination with the anti-adherent compounds, should not precipitate out, form a coacervate, or prevent a user from perceiving the conditioning benefit (or other desired benefit) to be gained from the composition. The thickening system may include a thickener which can provide both the thickening effect desired from the thickening system and a conditioning effect to the user's skin.

Thickeners may include, cellulosics, gums, acrylates, starches and various polymers. Suitable examples include but are not limited to hydroxethyl cellulose, xanthan gum, guar gum, potato starch, and corn starch. In some embodiments, PEG-150 stearate, PEG-150 distearate, PEG-175 diisostearate, polyglyceryl-10 behenate/eicosadioate, disteareth-100 IPDI, polyacrylamidomethylpropane sulfonic acid, butylated PVP, and combinations thereof may be suitable.

While the viscosity of the composition will typically depend on the thickener used and the other components of the composition, the thickeners of the composition suitably provide for a composition having a viscosity in the range of greater than 10 cP to about 30,000 cP or more. In another embodiment, the thickeners provide a composition having a viscosity of from about 100 cP to about 20,000 cP. In yet another embodiment, the thickeners provide a composition having a viscosity of from about 200 cP to about 15,000 cP.

Typically, the anti-adherent compositions of the present disclosure include the thickening system in an amount of no more than about 20% (by total weight of the composition), or from about 0.01% (by total weight of the composition) to about 20% (by total weight of the composition). In another aspect the thickening system is present in the anti-adherent composition in an amount of from about 0.05% (by total weight of the composition) to about 15% (by total weight of the composition), or from about 0.075% (by total weight of the composition) to about 10% (by total weight of the composition), or from about 0.1% (by total weight of the composition) to about 7.5% (by total weight of the composition).

### Emulsifiers

In one embodiment, the anti-adherent compositions may include hydrophobic and hydrophilic ingredients, such as when used in a lotion or cream. Generally, these emulsions have a dispersed phase and a continuous phase, and are generally formed with the addition of a surfactant or a combination of surfactants with varying hydrophilic/lipophilicbalances (HLB). Suitable emulsifiers include surfactants having HLB values from 0 to 20, or from 2 to 18. Suitable non-limiting examples include Ceteareth-20, CetearylGlucoside, Ceteth-10, Ceteth-2, Ceteth-20, Cocamide MEA, GlycerylLaurate, Glyceryl Stearate, PEG-100 Stearate, Glyceryl Stearate, Glyceryl Stearate SE, Glycol Distearate, Glycol Stearate, lsosteareth-20, Laureth-23, Laureth-4, Lecithin, Methyl Glucose Sesquistearate, Oleth-10, Oleth-2, Oleth-20, PEG-100 Stearate, PEG-20 Almond Glycerides, PEG-20 Methyl Glucose Sesquistearate, PEG-25 Hydrogenated Castor Oil, PEG-30 Dipolyhydroxystearate, PEG-4 Dilaurate, PEG-40 SorbitanPeroleate, PEG-60 Almond Glycerides, PEG-7 Olivate, PEG-7 GlycerylCocoate, PEG-8 Dioleate, PEG-8 Laurate, PEG-8 Oleate, PEG-80 SorbitanLaurate, Polysorbate 20, Polysorbate 60, Polysorbate 80, Polysorbate 85, Propylene Glycol Isostearate, Sorbitanlsostearate, SorbitanLaurate, SorbitanMonostearate, SorbitanOleate, SorbitanSesquioleate, Sorbitan Stearate, SorbitanTrioleate, Stearamide MEA, Steareth-100, Steareth-2, Steareth-20, Steareth-21. The compositions can further include surfactants or combinations of surfactants that create liquid crystalline networks or liposomal networks. Suitable non-limiting examples include OLIVEM 1000 (INCI: CetearylOlivate (and) SorbitanOlivate (available from HallStar Company (Chicago, Ill.)); ARLACEL LC (INCI: Sorbitan Stearate (and) SorbitylLaurate, commercially available from Croda (Edison, N.J.)); CRYSTALCAST MM (INCI: Beta Sitosterol (and) Sucrose Stearate (and) Sucrose Distearate (and) Cetyl Alcohol (and) Stearyl Alcohol, commercially available from MMP Inc. (South Plainfield, N.J.)); UNIOX CRISTAL (INCI: Cetearyl Alcohol (and) Polysorbate 60 (and) CetearylGlucoside, commercially available from Chemyunion (Sao Paulo, Brazil)). Other suitable emulsifiers include lecithin, hydrogenated lecithin, lysolecithin, phosphatidylcholine, phospholipids, and combinations thereof.

### Adjunct Ingredients

The anti-adherent compositions of the present disclosure may additionally include adjunct ingredients conventionally found in pharmaceutical compositions in their art-established fashion and at their art-established levels. For example, the anti-adherent compositions may comprise additional compatible pharmaceutically active and compatible materials for combination therapy, such as antioxidants, anti-parasitic agents, antipruritics, antifungals, antiseptic actives, biological actives, astringents, keratolytic actives, local anaesthetics, anti-stinging agents, anti-reddening agents, skin soothing agents, external analgesics, film formers, skin exfoliating agents, sunscreens, and combinations thereof.

Other suitable additives that may be included in the anti-adherent compositions of the present disclosure include compatible colorants, deodorants, emulsifiers, anti-foaming agents (when foam is not desired), lubricants, skin conditioning agents, skin protectants and skin benefit agents (e.g., aloe vera and tocopheryl acetate), solvents, solubilizing agents, suspending agents, wetting agents, pH adjusting ingredients (a suitable pH range of the composition can be from about 3.5 to about 8), chelators, propellants, dyes and/or pigments, and combinations thereof.

Another component that may be suitable for addition to the anti-adherent compositions is a fragrance. Any compatible fragrance may be used. Typically, the fragrance is presentin an amount from about 0% (by weight of the composition) to about 5% (by weight of the composition), and more typically from about 0.01% (by weight of the composition) to about 3% (by weight of the composition). In one desirable embodiment, the fragrance will have a clean, fresh and/or neutral scent to create an appealing delivery vehicle for the end consumer.

Organic sunscreens that may be present in the anti-adherent compositions include ethylhexylmethoxycinnamate, avobenzone, octocrylene, benzophenone-4, phenylbenzimidazole sulfonic acid, homosalate, oxybenzone, benzophenone-3, ethylhexyl salicylate, and mixtures thereof.

Antimicrobial agents may be added to the anti-adherent compositions. For example, suitable antimicrobials include biocides such as a short-chain alcohol,benzoalkonium chloride (“BAC”), didecyl dimethyl ammonium chloride (“DDAC”), and zeolite (“CWT-A”). Other possible antimicrobial agents include: isothiazolone, alkyl dimethyl ammonium chloride, a triazine, 2-thiocyanomethylthio benzothiazol, methylene bisthiocyanate, acrolein, dodecylguanidine hydrochloride, a chlorophenol, a quaternary ammonium salt, gluteraldehyde, a dithiocarbamate, 2-mercatobenzothiazole, para-chloro-meta-xylenol, silver, chlorohexidine, polyhexamthylenebiguanide, a n-halamine, triclosan, a phospholipid, an alpha hydroxyl acid, 2,2-dibromo-3-nitrilopropionamide, 2-bromo-2-nitro-1,3-propanediol, farnesol, iodine, bromine, hydrogen peroxide, chlorine dioxide, a botanical oil, a botanical extract, benzalkonium chloride, chlorine, sodium hypochlorite, or combinations thereof.

When present, the amount of the antimicrobial agent in the anti-adherent compositions is in an amount between about 0.01% to about 5% (by total weight of the composition), or in some embodiments between about 0.05 to about 3% (by total weight of the composition).

### Preservatives

The anti-adherent compositions may include various preservatives to increase shelf life. Some suitable preservatives that may be used in the present disclosure include, but are not limited to phenoxyethanol, capryl glycol, glycerylcaprylate, sorbic acid, gallic acid, benzoic acid, sodium benzoate, potassium sorbate, KATHON CG®, which is a mixture of methylchloroisothiazolinone and methylisothiazolinone, (available from Rohm & Haas); DMDM hydantoin (e.g., GLYDANT, available from Lonza, Inc., Fair Lawn, N.J.); EDTA and salts thereof; iodopropynylbutylcarbamate; benzoic esters (parabens), such as methylparaben, propylparaben, butylparaben, ethylparaben, isopropylparaben, isobutylparaben, benzylparaben, sodium methylparaben, and sodium propylparaben; 2-bromo-2-nitropropane-1,3-diol; and the like. Other suitable preservatives include those sold by Sutton Labs, such as “GERMALL 115” (imidazolidinyl urea), “GERMALL II” (diazolidinyl urea), and “GERMALL PLUS” (diazolidinyl urea and iodopropynylbutylcarbonate).

The amount of the preservative in the anti-adherent compositions is dependent on the relative amounts of other components present within the composition. For example, in some embodiments, the preservative is present in the compositions in an amount between about 0.001% to about 5% (by total weight of the composition), in some embodiments between about 0.01 to about 3% (by total weight of the composition), and in some embodiments, between about 0.05% to about 1.0% (by total weight of the composition).

### Preparation of Anti-Adherent Compositions

The anti-adherent compositions of the present disclosure may be prepared by combining ingredients at room temperature and mixing.

In one embodiment, when the anti-adherent composition is to be applied to the skin of an individual, the composition includes the anti-adherent agent, a hydrophilic carrier and a hydrophilic thickener. Suitable hydrophilic carriers can be, for example, water, glycerin, glycerin derivatives, glycols, water-soluble emollients, and combinations thereof. Suitable examples of glycerin derivatives could include, but are not to be limited to, PEG-7 glycerylcocoate. Suitable glycols could include, but are not to be limited to, propylene glycol, butylene glycol, pentylene glycol, ethoxydiglycol, dipropylene glycol, propanediol, and PEG-8. Suitable examples of water-soluble emollients could include, but are not to be limited to, PEG-6 CaprylicCapric Glycerides, Hydrolyzed Jojoba Esters, and PEG-10 Sunflower Glycerides.

### Delivery Vehicles

The anti-adherent compositions of the present disclosure may be used in combination with a product. For example, the composition may be incorporated into or onto a substrate, such as a wipe substrate, an absorbent substrate, a fabric or cloth substrate, a tissue substrate, or the like. In one embodiment, the anti-adherent composition may be used in combination with a wipe substrate to form a wet wipe or may be a wetting composition for use in combination with a wipe which may be dispersible. In other embodiments, the anti-adherent composition may be incorporated into wipes such as wet wipes, hand wipes, face wipes, cosmetic wipes, cloths and the like. In yet other embodiments, the anti-adherent compositions described herein can be used in combination with numerous personal care products, such as absorbent articles. Absorbent articles of interest are diapers, training pants, adult incontinence products, feminine hygiene products, and the like; bath or facial tissue; and paper towels. Personal protective equipment articles of interest include but are not limited to masks, gowns, gloves, caps, and the like.

In one embodiment, the wet wipe may comprise a nonwoven material that is wetted with an aqueous solution termed the “wetting composition,” which may include or be composed entirely of the anti-adherent compositions disclosed herein. As used herein, the nonwoven material comprises a fibrous material or substrate, where the fibrous material or substrate comprises a sheet that has a structure of individual fibers or filaments randomly arranged in a mat-like fashion. Nonwoven materials may be made from a variety of processes including, but not limited to, airlaid processes, wet-laid processes such as with cellulosic-based tissues or towels, hydroentangling processes, staple fiber carding and bonding, melt blown, and solution spinning.

The fibers forming the fibrous material may be made from a variety of materials including natural fibers, synthetic fibers, and combinations thereof. The choice of fibers may depend upon, for example, the intended end use of the finished substrate and the fiber cost. For instance, suitable fibers may include, but are not limited to, natural fibers such as cotton, linen, jute, hemp, wool, wood pulp, etc. Similarly, suitable fibers may also include: regenerated cellulosic fibers, such as viscose rayon and cuprammonium rayon; modified cellulosic fibers, such as cellulose acetate; or synthetic fibers, such as those derived from polypropylenes, polyethylenes, polyolefins, polyesters, polyamides, polyacrylics, etc. Regenerated cellulose fibers, as briefly discussed above, include rayon in all its varieties as well as other fibers derived from viscose or chemically modified cellulose, including regenerated cellulose and solvent-spun cellulose, such as Lyocell. Among wood pulp fibers, any known papermaking fibers may be used, including softwood and hardwood fibers. Fibers, for example, may be chemically pulped or mechanically pulped, bleached or unbleached, virgin or recycled, high yield or low yield, and the like. Chemically treated natural cellulosic fibers may be used, such as mercerized pulps, chemically stiffened or crosslinked fibers, or sulfonated fibers.

In addition, cellulose produced by microbes and other cellulosic derivatives may be used. As used herein, the term “cellulosic” is meant to include any material having cellulose as a major constituent, and, specifically, comprising at least 50 percent by weight cellulose or a cellulose derivative. Thus, the term includes cotton, typical wood pulps, non-woody cellulosic fibers, cellulose acetate, cellulose triacetate, rayon, thermomechanical wood pulp, chemical wood pulp, debonded chemical wood pulp, milkweed, or bacterial cellulose. Blends of one or more of any of the previously described fibers may also be used, if so desired.

The fibrous material may be formed from a single layer or multiple layers. In the case of multiple layers, the layers are generally positioned in a juxtaposed or surface-to-surface relationship and all or a portion of the layers may be bound to adjacent layers. The fibrous material may also be formed from a plurality of separate fibrous materials wherein each of the separate fibrous materials may be formed from a different type of fiber.

Airlaid nonwoven fabrics are particularly well suited for use as wet wipes. The basis weights for airlaid nonwoven fabrics may range from about 20 to about 200 grams per square meter (gsm) with staple fibers having a denier of about 0.5-10 and a length of about 6-15 millimeters. Wet wipes may generally have a fiber density of about 0.025 g/cc to about 0.2 g/cc. Wet wipes may generally have a basis weight of about 20 gsm to about 150 gsm. More desirably the basis weight may be from about 30 to about 90 gsm. Even more desirably the basis weight may be from about 50 gsm to about 75 gsm.

Processes for producing airlaid non-woven basesheets are described in, for example, published U.S. Pat. App. No. 2006/0008621, herein incorporated by reference to the extent it is consistent herewith.

The disclosure will be more fully understood upon consideration of the following non-limiting Examples.

### EXAMPLES

The following non-limiting examples show the efficacy of the anti-adherent compositions with respect to porcine skin and polystyrene controls and various microbes.

Table 2 shows the reduction of microbes on porcine skin after a two-hour adhesion time. The microbes tested were Staphylococus aureus, Klebsiella pneumonia, Candida albicans and T4 phage. Overall, only one extract, the Mum cepa extract, inhibited the attachment of all four microbes to porcine skin. With the exception of T4 phage, the Astragalus extract, Rhubarb root extract, Ginkgo biloba extract and Silymarin inhibited the attachment of the remaining three microbes to the porcine skin. Horse chestnut extract failed to inhibit the attachment of microbes to porcine skin and instead, attracted each of the four microbes. With respect to S. aureus, only Horse chestnut extract and Resveratrol failed to demonstrate anti-adherence. With respect to K. pneumonia, Soybean extract, Horse chestnut extract, Gypenoside and Resveratrol failed to demonstrate anti-adherence. With respect to C. albicans, Horse chestnut extract and Cassia seed extract failed to demonstrate anti-adherence. Finally, with respect to T4 phage, all botanical agents except Resveratrol and Allium cepa extract failed to demonstrate anti-adherence.

Table 3 shows the percentage of microbes that remain attached to treated polystyrene surfaces after one-hour adhesion time. The microbes tested were S. aureus, K. pneumonia, and C. albicans. Overall, with the exception of Ginkgo biloba extract and Astragalus extract, all extracts inhibited the attachment of all three microbes after a one-hour adhesion time. Cassia seed extract was the most anti-adherent to all three microbes, followed by Gypenoside. If only S. aureus, K. pneumonia is of interest, then Horse chestnut extract, Allium cepa extract, Cassia seed extract, and Gingko biloba extract are the most effective anti-adherent agents and almost equal in efficacy.

Table 4 shows the percentage of microbes that remain attached to treated polystyrene surfaces after fifteen-minute adhesion time. The microbes tested were S. aureus, and E. coli. Of the eight botanical agents tested, only two demonstrated anti-adherence with respect to the microbe, Astragalus extract and Gingko biloba extract. All the extracts tested were anti-adherent with respect to S. aureus except for Rhubarb root extract and Silymarin. Only three extracts tested were anti-adherent with respect to E. coli, Astragalus extract, Rhubarb root extract, and Ginkgo biloba extract.

Because the test results varied depending on the microbe and type of surface tested, there is no single botanical agent that is anti-adherent in all circumstances. However, one botanical agent does appear to offer a broad spectrum of protection against microbe adherence to both tested surfaces. Specifically, if one is interested in anti-adherent compositions for the skin and a hard surface like polystyrene, it may be that Allium cepa extract is the best choice for a broad spectrum of protection as it was only adherent to E. coli on polystyrene. If one is only concerned about skin, Allium cepa extract may offer the broadest protection against the adherence of microbes. If one is interested in anti-adherent compositions for surfaces having characteristics similar to polystyrene, then Allium cepa extract may still be of interest if S. aureus and K. pneumonia are the microbes of concern. If S. aureus is the only microbe of concern, then Gypenoside, Astragalus extract, Allium cepa extract, Ginkgo biloba extract, and Cassia seed extract may be the best choices for an anti-adherent composition.

### Example 1

The botanical agents listed in Table 2 were initially screened for their effect against microbial adhesion using the Test Methods below. The effect of the botanical agents as the percentage of control-corrected A490 is shown. For bacterial strains, the corrected A490 were all less than 20% of the untreated control. For C. albicans, the corrected A490 of the botanical filtrates were less than 70% of the untreated control. The higher percentage value indicates a stronger inhibitory effect

Table 2 shows the reduction of microbes on porcine skin after a 2 hour adhesion time was allowed. Positive numbers indicate anti-adherent compounds/extracts.

| | | Reduction of AdherentCells (%) from Porcine Skin Control* | | | |
|---|---|---|---|---|---|
| Botanical (Bot.) | Bot. | (Two-Hour Adhesion Time) | | | |
| Agents | (%) | S. aureus | K. pneumoniae | C. albicans | T4 phage |
| Soybean extract | 5 | 40.6 ± 8.3 | −325.3 ± 207.9 | 23.6 ± 7.6 | −19 ± 57.3 |
| Horse chestnut extract | 5 | −30.2 ± 39.7 | −64.4 ± 21.1 | −2.7 ± 4.6 | −40.5 ± 18.0 |
| Gypenoside | 5 | 39.2 ± 17.8 | −23.0 ± 24.9 | 19.6 ± 18.7 | −61.9 ± 32.2 |
| Resveratrol | 5 | −4.2 ± 19.1 | −120.7 ± 95.8 | 29.8 ± 11.2 | 159.5 ± 111.2 |
| Astragalus extract | 5 | 20.8 ± 25.3 | 50.0 ± 18.0 | 22.2 ± 45.4 | −104.8 ± 113.9 |
| Rhubarb root extract | 5 | 57.3 ± 11.0 | 24.3 ± 36.0 | 50.7 ± 7.1 | −9.6 ± 41.7 |
| Allium cepa extract | 1.25 | 44.1 ± 21.2 | 42.9 ± 36.7 | 11.1 ± 38.7 | 17.8 ± 20.4 |
| Cassia seed extract | 0.312 | 59.8 ± 6.9 | 29.3 ± 22.7 | −17.0 ± 54.1 | −46.7 ± 78.6 |
| Ginkgo biloba extract | 0.625 | 31.9 ± 26.2 | 46.7 ± 31.1 | 43.8 ± 14.5 | −93.3 ± 86.7 |
| Silymarin | 0.312 | 27.9 ± 36.1 | 12.4 ± 46.8 | 13.1 ± 13.1 | −55.6 ± 73.7 |

*(Average +/− SD, n = 2)
Bot. Wt. % = Concentration of Agent in water by total weight of solution, percent

### Example 2

The botanical extracts listed in Table 3 were initially screened for their effect against microbial adhesion using the High Throughput Anti-adherent Test Method described below. The effect of the botanical filtrates as the percentage of control-corrected A490 is shown. For bacterial strains, the corrected A490 were all less than 20% of the untreated control. For C. albicans, the corrected A490 of the botanical filtrates were less than 70% of the untreated control. The lower percentage value indicates a stronger inhibitory effect.

TABLE 3
The percentage of microbes that remain attached to treated polystyrene surfaces after one-hour
adhesion time. A low percentage value is indicative of a stronger anti-adherent effect.
For practical purposes, the bracketed numbers indicate an adherent effect.

| | Reduction of Adherent Cells (%) from | | |
|---|---|---|---|
| | Polystyrene Control** | | |
| Botanical | (One-Hour Adhesion Time) | | |
| Agents | S. aureus | K. pneumoniae | C. albicans |
| Soybean extract | 3.0 ± 4.2 | <1 | 19* |
| Horse chestnut extract | <1 | <1 | 36* |
| Gypenoside | 5.5 ± 7.8 | 5.0 ± 7.1 | 13.5 ± 7.8 |
| Resveratrol | 8.0 ± 9.9 | 7.0 ± 2.8 | 25.5 ± 4.9 |
| Astragalus extract | 10.5 ± 4.9 | 5 ± 1.4 | [164.5 ± 29.0] |
| Rhubarb root extract | 8.5 ± 12.0 | 15.5 ± 12.0 | 16* |
| Allium cepa extract | <1 | <1 | 29.0 ± 7.1 |
| Cassia seed extract | <1 | <1 | 1.5 ± 2.1 |
| Ginkgo biloba extract | <1 | <1 | [59.0 ± 19.8] |
| Silymarin | 15.5 ± 3.5 | 7.5 ± 9.2 | 67* |

*Results of a single measurement
**(Average +/− SD, n = 2)

Botanical Agents=5% Concentration of Agent in water by total weight of solution, percent

### Example 3

The botanical agents listed in Table 4 were initially screened for their effect against microbial adhesion using the High Throughput Anti-adherent Test Method described below.

Table 4 shows the reduction of microbes as compared to the controls on polystyrene MBEC surfaces after a 15-minute adhesion time. Bracketed numbers indicate anti-adherent results.

| | Reduction of AdherentCells (Log) | |
|---|---|---|
| | from Polystyrene Control* | |
| Botanical | (15-Minute Adhesion Time) | |
| Agents | S. aureus ATCC 6538 | E. coli ATCC 11229 |
| Horse chestnut extract | [0.9 ± 0.3] | −0.1 ± 0.1 |
| Gypenoside | [2.1 ± 0.2] | 0.1 ± 0.6 |
| Astragalus extract | [1.1 ± 0.3] | [0.6 ± 0.2] |
| Rhubarb root extract | 0.5 ± 0.3 | [0.8 ± 0.3] |
| Allium cepa extract | [1.2 ± 0.2] | 0.3 ± 0.5 |
| Cassia seed extract | [1.3 ± 0.2] | −0.6 ± 0.3 |
| Ginkgo biloba extract | [0.9 ± 0.1] | [0.8 ± 0.6] |
| Silymarin | 0.5 ± 0.2 | −0.1 ± 0.6 |

*(Average +/− SD, n = 2)
Botanical agents = 5% Concentration of Agent in water by total weight of solution, percent

### Test Methods

I. Screening on Porcine Skin

Preparation of Bacterial Cell Suspensions
1. Klebsiella pneumonia CICC 21519 (ATCC 4352) and Staphylococcus aureus CICC 10384 (ATCC 6538P) were purchased from China Center of Industrial Culture Collection.
2. Stock culture of each species was streaked onto TSA plate (OXOID, HAMPSHIRE, England) and incubated at 37° C. overnight.
3. Single colonies were subcultured in TSB (OXOID, HAMPSHIRE, England) and incubated 37° C., 200 rpm overnight.
4. Dilutions of overnight cultures in PBS buffer were used for the screening.

Preparation of C. albicans Cell Suspension
1. Single cell suspension of C. albicans SC5314 was prepared as follows. Stock culture of C. albicans SC5314 was streaked onto YPD agar (YPDA) and incubated at 30° C. overnight.
2. Single colonies were subcultured in YPD broth (YPDB) and incubated 30° C., 200 rpm overnight.
3. Overnight culture in YPDB was subcultured in YPD broth again and incubated 30° C., 200 rpm for 48 hours.
4. Cells were collected by centrifugation (4000×g, 10 minutes) at 4° C., washed three times with sterile water, and then resuspended in sterile water a final concentration of 10\hat{ }9 CFU/mL.
5. The suspension were stored at 4° C. at least 1 day, then subcultured into YPDB to a final concentration of 10\hat{ }6 CFU/mL.
6. After 48 hours incubation, cell suspension was examined under microscope for single cell percentage.
7. When the single cell percentage exceeded 80%, cells were collected and washed three times with water, then resuspended in water to a final concentration of 10\hat{ }9 CFU/mL, and stored at 4° C. prior to use.
8. The suspension was diluted in PBS for screening anti-adhesion compounds.

Preparation of T4 Phage Suspension
1. T4 phage (Research Center for Eco-Environmental Sciences, Chinese Academy of Science, Beijing) suspension was prepared as follows. A single colony was inoculated into 5 mL LB broth and incubated at 37° C., 200 rpm overnight.
2. The overnight culture (0.5 mL) was subcultured into LB broth and incubated at 37° C., 200 rpm for 6 hours.
3. The 6 hour host culture (2 mL) was mixed with 2 mL of T4 stock and 10 mL LB broth, and incubated at 37° C.
4. After overnight incubation, the broth was centrifuged (6000 rpm, 10 minutes, 4° C.). The supernatant was collected and filtered using 0.20 μm filters (Millipore, CORK, IRELAND), and stored at 4° C.

Screening the Anti-Adhesion Effect of Botanicals/Compounds on Porcine Skin
1. Porcine skins were aseptically cut to squares of 2×2 cm, and each was placed in a sterile Petri dish (CITOTEST LABWARE MANUFACTURING CO., LTD, Haimen, Jiangsu).
2. Aliquots (100 μL) of botanical/compounds suspensions were applied to porcine skins, three replicates.
3. Skins were allowed to air dry for 2 hours at ˜50% relative humidity.
4. Inocula (100 μL) were applied to the porcine skin, and incubated at 37° C. for 2 hours.
5. The skins were washed three times by applying 5 ml of PBS+0.01% Tween 80 onto the skins and then removing the liquid by pipetting.
6. The skins were then placed in 50 mL centrifuge tube (CITOTEST LABWARE MANUFACTURING CO., LTD, Haimen, Jiangsu) with 10 mL of PBS, and the adhered cells were washed off by sonication for 5 minutes (1 minute on and 1 minute off), and enumerated.
7. Microbial cells were enumerated by spread plate method.
   a. For C. albicans, the suspensions and dilutions were plated onto YPD, and incubated at 25° C. for 2 days.
   b. For S. aureus and K. pneumoniae, the suspension and dilutions were plated onto TSA, and incubated at 37° C. for 1 day.
8. After incubation, colonies on plates were counted. Reduction (%) of colony forming unit (CFU) from the average counts on untreated skin was calculated.
9. T4 phage were enumerated by double layer agar method.
   a. Aliquots (100 μL) of suspensions and dilutions were mixed with in 2.5 ml LB soft agar (LBS,LB broth with 0.5% agar) with 50 μL of 4 hour culture of E. coli B (Research Center for Eco-Environmental Sciences, Chinese Academy of Science, Beijing), and poured over pre-warmed LB agar plates (45° C., LB broth with 1.5% agar).
   b. The plates were left at room temperature until set, and incubated at 37° C. overnight.
   c. After incubation, plaques on plates were counted.
   d. Reduction (%) of plaque forming unit (PFU) from the average counts on untreated skin was calculated.

Media

YPD Agar
Peptone (OXOID, HAMPSHIRE, England) 20.0 g
Yeast extract (OXOID, HAMPSHIRE, England) 10.0 g
Glucose (Sinopharm Chemical Reagents, Shanghai) 20.0 g
Agar (Sunshine Biotechnology, Nanjing, Jiangsu) 15.0 g
Milli-Q water 1.0 L
Mix all the ingredients, sterilize by autoclave at 115° C. for 30 minutes.

YPD Broth
Peptone (OXOID, HAMPSHIRE, England) 20.0 g
Yeast extract (OXOID, HAMPSHIRE, England) 10.0 g
Glucose (Sinopharm Chemical Reagents, Shanghai) 20.0 g
Milli-Q water 1.0 L
Mix all the ingredients, sterilize by autoclave at 115° C. for 30 minutes.

PBS Buffer (pH 7.0)
NaCl (Sinopharm Chemical Reagents, Shanghai) 9 g
Na2HPO4.12H2O (Sinopharm Chemical Reagents, Shanghai) 3.44 g
NaH2PO4.2 H₂O (Sinopharm Chemical Reagents, Shanghai) 0.243 g
Milli-Q water 1 L
Dissolve all reagents, adjust pH to pH 7.0, sterilize by autoclave at 121° C. for 30 minutes.

PBS Buffer with 0.01% Tween 80 (PBS-T, pH 7.0)
NaCl (Sinopharm Chemical Reagents, Shanghai) 9 g
Na2HPO4. (Sinopharm Chemical Reagents, Shanghai) 12H2O 3.44 g
NaH2PO4.2 H₂O (Sinopharm Chemical Reagents, Shanghai) 0.243 g
Tween 80 (Sinopharm Chemical Reagents, Shanghai) 0.05 g
Milli-Q water 500 mL
Dissolve all reagents, adjust pH to pH 7.0, sterilize by autoclave at 121° C. for 30 minutes.

LB Broth
Tryptone (OXOID, HAMPSHIRE, England) 10.0 g
Yeast extract (OXOID, HAMPSHIRE, England) 5.0 g
NaCl (Sinopharm Chemical Reagents, Shanghai) 10.0 g
Milli-Q water 1.0 L
Mix all the ingredients, sterilize by autoclave at 121° C. for 30 minutes.

II. High Throughput Anti-Adherence Test Method

This test method specifies the operational parameters required to grow and or prevent the formation of bacterial attachment using a high throughput screening assay. The assay device consists of a plastic lid with ninety-six (96) pegs and a corresponding receiver plate with ninety-six (96) individual wells that have a maximum 200 μL working volume. Biofilm is established on the pegs under static batch conditions (i.e., no flow of nutrients into or out of an individual well).
1. Terminology
   1.2 Definitions of Terms Specific to This Standard:
   1.2.2 peg, n—biofilm sample surface (base: 5.0 mm, height: 13.1 mm).
   1.2.3 peg lid, n—an 86×128 mm plastic surface consisting of ninety-six (96) identical pegs.
   1.2.4 plate, n—an 86×128 mm standard plate consisting of ninety-six (96) identical wells.
   1.2.5 well, n—small reservoir with a 50 to 200 μL working volume capacity.
2. Acronyms
   2.2 ATCC: American Type Culture Collection
   2.3 CFU: colony forming unit
   2.4 rpm: revolutions per minute
   2.5 SC: sterility control
   2.6 TSA: tryptic soy agar
   2.7 TSB: tryptic soy broth
   2.8 GC: growth control
3. Apparatus
   3.2 Inoculating loop—nichrome wire or disposable plastic.
   3.3 Petri dish—large labelled (100×150×15 mm, plastic, sterile) for plating.
   3.4 Microcentrifuge tubes—sterile, any with a 1.5 mL volume capacity.
   3.5 96-well microtiter plate—sterile, 86×128 mm standard plate consisting of ninety-six (96) identical flat bottom wells with a 200 μL working volume
   3.6 Vortex—any vortex that will ensure proper agitation and mixing of microfuge tubes.
   3.7 Pipette—continuously adjustable pipette with volume capability of 1 mL.
   3.8 Micropipette—continuously adjustable pipette with working volume of 10 μL-200 μL.
   3.9 Sterile pipette tips-200 uL and 1000 uL volumes.
   3.10 Sterile reagent reservoir-50 mL polystyrene.
   3.11 Sterilizer—any steam sterilizer capable of producing the conditions of sterilization.
   3.12 Colony counter—any one of several types may be used. A hand tally for the recording of the bacterial count is recommended if manual counting is done.
   3.13 Environmental incubator—capable of maintaining a temperature of 35±2° C. and relative humidity between 35 and 85%.
   3.14 Reactor components—the MBEC Assay device available from Innovotech, Edmonton, AB, Canada.
   3.15 Sterile conical tubes—50 mL, used to prepare initial inoculum.
   3.16 Appropriate glassware—as required to make media and agar plates.
   3.17 Erlenmeyer flask—used for growing broth inoculum.
   3.18 Positive Displacement pipettes capable of pipetting 200 μL.
   3.19 Sterile pipette tips appropriate for Positive Displacement pipettes.
4. Reagents and Materials
   4.2 Purity of water—all references to water as diluent or reagent shall mean distilled water or water of equal purity.
   4.3 Culture media:
   4.4 Bacterial growth broth—Tryptic soy broth (TSB) prepared according to manufacturer's directions.
   4.5 Bacterial plating medium—Tryptic soy agar (TSA) prepared according to manufacturer's directions.
   4.6 Phosphate Buffered Saline (PBS)—
   4.7 Rinse Solution: Sterile PBS and Tween 80 1% w/v.
5. Microorganisms:
   5.1 E. coli ATCC 11229 and S. aureus ATCC 6538
6. TEST METHOD overview: The experimental process for the High-Throughput Anti-Adherence Test Method. This standard protocol may be broken into a series of small steps, each of which is detailed in the sections below.
   6.1 Culture Preparation
   6.1.1 E. coli ATCC 11229 and S. aureus ATCC 6538 are the organisms used in this test.
   6.1.2 Using a cryogenic stock (at −70° C.), streak out a subculture of the above listed microorganisms on organism's specific agar (TSA).
   6.1.3 Incubate at 35±2° C. for the period of time of 22±2 hours.
   6.1.4 9.1.4 Aseptically remove isolated colony from streak plate and inoculate 20 mL of sterile TSB.
   6.1.5 Incubate flask at 35±2° C. and 175±10 rpm for 16 to 18 hours (E. coli and S. aureus). Viable bacterial density should be 10⁹ CFU/mL and should be checked by serial dilution and plating.
   6.1.6 Pipette 10 mL from the incubation flask of E. coli and S. aureus into a 50 mL conical tube and spin down at 5 minutes at 4,000×g. Then remove supernatant and Resuspend in 10 mL sterile PBS. Approximate cell density should be 10⁷-10⁹ CFU/mL. Vortex the sample for approximately 30 seconds to achieve a homogeneous distribution of cells.
   6.1.7 Perform 10-fold serial dilutions of the inoculum in triplicate.
   6.1.8 Plate appropriate dilutions on appropriately labelled TSA plates. Incubate the plates at 35±2° C. for 22±2 hours depending on the isolates growth rate and enumerate.
   6.2 Preparation of the Challenge plates:
   6.2.1 Preparation of compounds and coating compounds onto MBEC plate lid
   6.2.1.1.1 Using a positive displacement pipette aseptically add 200 μL of compounds and control to be tested to a sterile 96-well microplate according to the plate layout of Table 5.

TABLE 5
Sample layout of 96-well MBEC plate.

| | | 1 | 2 | 3 | 4 | 5 | 6 | 7 | 8 | 9 | 10 | 11 | 12 |
|---|---|---|---|---|---|---|---|---|---|---|---|---|---|
| E. coli | A | AAC | | T1 | T2 | T3 | T4 | T5 | T6 | T7 | T8 | NT-GC | T1-SC |
| E. coli | B | AAC | | T1 | T2 | T3 | T4 | T5 | T6 | T7 | T8 | NT-GC | T2-SC |
| E. coli | C | AAC | | T1 | T2 | T3 | T4 | T5 | T6 | T7 | T8 | NT-GC | T3-SC |
| E. coli | D | AAC | | T1 | T2 | T3 | T4 | T5 | T6 | T7 | T8 | NT-GC | T4-SC |
| S. aureus | E | AAC | | T1 | T2 | T3 | T4 | T5 | T6 | T7 | T8 | NT-GC | T5-SC |
| S. aureus | F | AAC | | T1 | T2 | T3 | T4 | T5 | T6 | T7 | T8 | NT-GC | T6-SC |
| S. aureus | G | AAC | | T1 | T2 | T3 | T4 | T5 | T6 | T7 | T8 | NT-GC | T7-SC |
| S. aureus | H | AAC | | T1 | T2 | T3 | T4 | T5 | T6 | T7 | T8 | NT-GC | T8-SC |

AAC = Anti-Adherent Control
NT-GC = No Treatment Growth Control
SC = Sterility Control
T1-T8 = Test Codes

6.2.1.1.2 Add 200 μL of each code to the appropriate well for sterility controls.
6.2.1.1.3 Place the MBEC plate lid, peg side down into the 96-well microplate containing the test compound solutions.
6.2.1.1.4 Allow the plate to sit at room temperature (25±3° C.) for 2 hours.
6.2.1.1.5 Remove the MBEC plate lid and allow the lid to dry at room temperature (25±3° C.) overnight in a laminar flow hood.
7.1 Bacterial Adherence Challenge:
   7.1.1 Add 100 μL of diluted bacteria to the appropriate wells in a sterile 96-well microplate as indicated in the plate layout in Table 5.
   7.1.2 Add 200 μL of sterile PBS to the sterility controls.
   7.1.3 The MBEC containing dried compounds is then inserted into the bacterial inoculated 96 well flat bottom microplate from section 9.3.1
   7.1.4 Incubate stationary at room temperature (25±3° C.) for 15 minutes.
   7.1.5 Remove the MBEC lid and place into a 96-well microplate containing 200 μL PBS+1% w/v Tween-80. Incubate stationary at room temperature (25±3° C.) for 15 seconds.
   7.1.6 Repeat step 7.1.5 for two additional washes for a total of 3 washes.
7.2 Method to Determine Number of Attached Bacteria
   7.2.1 Transfer the washed MBEC plate lid to a 96-well plate containing 200 μL ALAMARBLUE reagent (prepared according to manufacturer's directions, Life Technologies, Carlsbad, Calif.) in each well to be tested.
   7.2.2 The final plate is transferred to a SPECTRAMAX GEMINI EM microplate reader (Molecular Devices, Inc. Sunnyvale, Calif. USA) for a 20 hour kinetic, bottom read with an excitation of 560 nm and emission of 590 nm. The rate of fluorescence development (relative fluorescence units (RFU)/minute) is determined for each well.
   7.2.3 Data was analyzed using a standard curve (described below) for each organism to determine the numbers of bacteria attached to the pegs (Log CFU/mL) present in each sample. Number of attached bacteria was quantified by incubating with an ALAMARBLUE reagent and measuring fluorescence development over time.
   7.2.4 From these data, the Log CFU/mL reduction of each time point relative to the growth control is calculated to determine the activity of each code.
7.3 Method for Generating a Standard Curve with bacteria in an ALAMARBLUE Solution:
   7.3.1 Standard curves were constructed for each organism to define the rate of fluorescence development as a function of bacterial concentration, as determined via viable plate counts. This standard curve provided the ability to relate rate of fluorescence development (RFU/minute) to the Log CFU/mL number of bacteria present in a given sample
   7.3.2 Day 1:
   7.3.2.1 Aseptically remove loopful of bacteria strain to be tested from freezer stock and place in 20 mL of TSB media in a culture flask.
   7.3.2.2 Incubate with shaking (200 rpm) for 22±2 hours at 37±2° C.
   7.3.3 Day 2:
   7.3.3.1 Aseptically transfer 100 μL of the 22±2 hours freezer stock cultures into 20 mL of TSB media in a culture flask.
   7.3.3.2 Incubate cultures on a gyrorotary shaker (200 rpm) for 22±2 hours at 37±2° C.
   7.3.3.3 Perform a streak for isolation from the culture flask on TSA. Incubate plate for 22±2 hours at 37±2° C.
   7.3.4 Day 3:
   7.3.4.1 Prepare an ALAMARBLUE solution according to the manufacturers directions.
   7.3.4.2 Remove culture flask from shaking incubator after 22±2 hours. Pipette 1 mL of bacteria into a 1.7 mL microcentrifuge tube.
   7.3.4.3 Centrifuge the bacteria at 4000×g.
   7.3.4.4 Resuspend bacterial cells in sterile PBS. Perform a total of two washes.
   7.3.4.5 Perform 1:10 serial dilutions with washed bacterial culture in 0.9 mL dilution blanks of sterile PBS (100 μL culture into 900 μL of sterile PBS).
   7.3.4.6 Plate appropriate dilutions of prepared bacteria.
   7.3.4.7 Add 270 μL of ALAMARBLUE solution to wells A-D: columns 1-7 of a 96-well plate.
   7.3.4.8 Add 30 μL of bacterial dilution the wells of a 96-well plate (n=4 per dilution).
   7.3.4.9 Add 30 μL of sterile PBS to wells A-D, column 8 for a background control.
   7.3.4.10 Place plate in a bottom reading spectrophotometer that measures fluorescence. Set temp to 37° C. Perform assay at 37° C., read every 20 minutes for 24 hours at 560 excite and 590 emit.
   7.3.4.11 Enumerate the dilutions.
   7.3.4.12 Calculate the mean rate of fluorescence development.
   7.3.4.13 Plot the mean rate of fluorescence development as a function of the mean CFU/mL of the dilutions.

When introducing elements of the present disclosure, the articles “a”, “an”, “the” and “said” are intended to mean that there are one or more of the elements. The terms “comprising”, “including” and “having” are intended to be inclusive and mean that there may be additional elements other than the listed elements. Many modifications and variations of the present disclosure can be made without departing from the spirit and scope thereof. Therefore, the exemplary embodiments described above should not be used to limit the scope of the invention.

1.-19. (canceled)

20. A method of inhibiting the attachment of a microbe to a surface, the method comprising:
providing an anti-adherent composition, the anti-adherent composition comprising:
a carrier; and
an effective amount of botanical agent selected from the group consisting of Soybean extract, Horse chestnut extract, Gypenoside, Resveratrol, Astragalus extract, Rhubarb root extract, Allium cepa extract, Cassia seed extract, Ginkgo biloba extract, Silymarin, and combinations thereof;
applying the composition to the surface; and
allowing at least some of the composition to remain on the surface such that the anti-adherent agent inhibits microbes from attaching to the surface.

21. The method of claim 20, wherein allowing at least some of the composition to remain on the surface such that the anti-adherent agent inhibits microbes from attaching to the surface reduces the attachment of the microbes to the surface by at least 0.5 Log of bacteria.

22. The method of claim 21, wherein the surface is polystyrene and the anti-adherent agent reduces the attachment of the microbe to the surface by at least 1 Log of bacteria.

23. The method of claim 22, wherein the botanical agent is Allium cepa extract.

24. The method of claim 23, wherein the microbe is Gram-negative bacteria, Gram-positive bacteria, yeast, or virus and the surface is porcine skin.

25. The method of claim 21, wherein the botanical agent is selected from the group consisting of Astragalus extract, Rhubarb root extract, Ginkgo biloba extract, Silymarin, and combinations thereof.

26. The method of claim 25, wherein the microbe is S. aureus, K. pneumonia, C. albicans, or T4 phage and the surface is porcine skin.

27. The method of claim 21, wherein the botanical agent is selected from the group consisting of Allium cepa extract, Cassia seed extract, Ginkgo biloba extract, Horse chestnut extract, and combinations thereof.

28. The method of claim 27, wherein the microbe is S. aureus or K. pneumonia and the surface is polystyrene.

29. The method of claim 20, wherein the at least some of the composition remains on the surface for at least fifteen minutes to inhibit microbes from attaching to the surface.

30. The method of claim 20, wherein the at least some of the composition remains on the surface for at least one hour to inhibit microbes from attaching to the surface.

31. The method of claim 20, wherein the at least some of the composition remains on the surface for at least two hours to inhibit microbes from attaching to the surface.

32. The method of claim 20, wherein the anti-adherent agent is present in the amount of 0.01% to 20% by weight of the composition.

33. The method of claim 20, wherein the carrier is hydrophilic.

34. The method of claim 20, wherein the composition further comprises a humectant selected from the group consisting of glycerin, hyaluronic acid, betaine, amino acids, glycosaminoglycans, glycols, polyols, sugars, sugar alcohols, hydrogenated starch hydrolysates, hydroxy acids, salts of pyrrolidone carboxylic acid, and combinations thereof.

35. The method of claim 34, wherein the humectant is selected from the group consisting of honey, sorbitol, hyaluronic acid, sodium hyaluronate, betaine, lactic acid, citric acid, sodium citrate, glycolic acid, sodium glycolate, sodium lactate, urea, propylene glycol, butylene glycol, pentylene glycol, ethoxydiglycol, methyl gluceth-10, methyl gluceth-20, PEG-2, PEG-3, PEG-4, PEG-5, PEG-6, PEG-7, PEG-8, PEG-9, PEG-10, xylitol, maltitol, and a combination thereof.

36. The method of claim 20, wherein the composition further comprises an emollient.

37. The method of claim 20, wherein the composition further comprises an emulsifier.

38. The method of claim 20, wherein the composition further comprises an antimicrobial agent.

39. The method of claim 20, wherein the composition is incorporated in a wipe, the wipe being used to apply the composition to the surface.
